(19) United States
(12) Patent Application Publication
(10) Pub. No.: US 2010/0248316 A1
(43) Pub. Date: Sep. 30, 2010

(54) Process for the Production of Ambrafuran

(75) Inventors: Lucia H. Steenkamp, Boksburg (ZA); Mihloti Taka, Gauteng (ZA)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: CSIR, Gauteng (ZA)

(21) Appl. No.: 12/726,559

(22) Filed: Mar. 18, 2010

(30) Foreign Application Priority Data
Mar. 25, 2009 (ZA) 2009/02099

(51) Int. Cl. C12P 17/04; C07D 307/92

(52) U.S. Cl. 435/126; 549/458

(57) ABSTRACT

A method for the cyclodehydration of a 1,4- or 1,5-diol includes the step of exposing a 1,4- or a 1,5-diol to an activated zeolite at a temperature of between about 0° C. and about 110° C. for a period of between about 1 and 24 hours. The activated zeolite is prepared from an inactive NaY or CaY type zeolite by ion exchange with an ammonium salt, to produce an ammonium zeolite and exchange of at least part of the ammonia of the ammonium zeolate with a group II A metal.

This application claims the benefit of priority of South African Provisional Patent Application No. 2009/02099, filed on Mar. 25, 2009, the disclosure of which is incorporated herein by reference in its entirety.

THIS INVENTION relates to the dehydration of alcohols. It relates in particular to the cyclodehydration of dials and to a process for the production of ambrafuran.

The food, feed, cosmetic, chemical and pharmaceutical sectors make extensive use of flavours and fragrances. Although many commercially available flavour compounds are produced via chemical synthesis or through extraction from plant and animal sources, there is a movement to produce these active compounds via bio-production which includes fermentation or bio-conversions using biocatalysts. The reason for this is partly because of consumer demand for “green products” which are manufactured by environmentally friendly chemical processes and partly because normal synthetic processes generally produce racemic mixtures instead of single enantiomers. The isolation of active compounds from plant and animal sources also usually has the drawback that these compounds are present in small quantities and this results in expensive processes.

For centuries, ambergris has been a very valuable perfumery material and has been used as a fixative agent in perfumes. A fixative agent, which can be a natural or a synthetic compound, reduces the rate of evaporation of volatile substances in perfumes and stabilises perfumes. Ambergris is a metabolic product produced by the sperm whale (Physeter macrocephatus L.). Ambergris is formed in the rectum of the whale from indigestible objects from the animals on which it feeds. These usually include the beaks of squid and cuttlefish, and the ambergris is normally released when the whale dies. Ambergris contains a large amount of steroid lipids and has a lower density than water. Following initial release, the ambra which is a pathological metabolite of the sperm whale is soft and pale white and has a strong manure smell. During exposure to the elements at sea, the ambra is oxidised and it loses the strong offensive smell and the characteristic ambergris odour develops. The material (−)-ambrafuran is the most important and sought-after of the compounds of the ambergris type and is marketed by Firmenich S. A under the trade mark Ambrox®. The literature names for (−)-ambrafuran are dodecahydro-3a,6,6,9a-tetramethylnaphtho[2,1-b]furan or (−)-8,12-epoxy-13,14,15,16-tetranorlabdane.

Several routes have been developed to produce (−)-ambrafuran synthetically and many are based on naturally occurring sesqui- or diterpenes. Sclareol has been used in industrial processes for the synthesis of (−)-ambrafuran as it has the same stereochemical features as (−)-ambrafuran. It has been reported that any changes in the configuration of the four chiral carbon atoms of ambrafuran have major effects on the odour, the profile and the strength of the compound.

The synthesis of (−)-ambrafuran from sclareol in an overall yield of approximately 76% has been reported and describes some of the problems encountered with the synthesis of ambrafuran from sclareol including the hemisynthesis of ambrafuran from sclareol which was carried out in three stages and in eight steps. These involved the oxidative degradation of the sclareol side chain to yield sclareolide and its acetoxy-acid precursor, followed by the reduction of the intermediate compounds to ambradiol and a final cyclodehydration to yield (−)-ambrafuran. The first stages of the synthesis appear to be problematic as three intermediates have to be formed successively. Potassium permanganate (7 moles per mole sclareol) was normally used for the oxidation of sclareol and only gave a yield of 65% sclareolide following saponification and lactonisation of the intermediate acetoxy-acid. A by-product of the reaction, was the production of manganese dioxide in quantities almost double the weight of the sclareol. This was a sticky solid and which was very difficult to remove. To overcome this problem, the manganese dioxide was converted to water-soluble manganese salts by reduction under acidic conditions using sulphur dioxide, sodium hydrogen sulfite and oxalic acid. This however resulted in aqueous waste disposal problems. Ruthenium tetraoxide (normally prepared in situ from ruthenium chloride), used in combination with common oxygen donors, is also used for catalytic oxidation. Prior art methods include the reaction of sclareol with ruthenium chloride hydrate (0.023 mol/mol) and sodium (˜8 periodate mol/mol) under typical Sharpless conditions to give a mixture of the acetoxy-acid and sclareolide in an 88.5% overall yield. In another prior art method the sodium periodate and organic solvent were replaced with sodium hypochlorite and water respectively and the yield of the sclareolide after saponification and lactonisation of the acetoxy-acid was 75-78%.

Other researchers have described the conversion of ambradiol to (−)-ambrafuran by ring closure through cyclodehydration. It has been shown that if the ring closure involves the attack on the 12-carbon by the 8-oxygen, the configuration of the 8-chiral carbon should be retained. This normally happens when the common method for preparation of cyclic ethers from 1,4-diols using tosyl chloride in pyridine is used. The reaction involves displacement of the tosyloxy group, which is selectively formed from the primary 12-hydroxyl, by the 8-oxygen in a nucleophilic substitution process. It may however be advantageous to avoid the use of pyridine entirely since, even at low levels, pyridine may interfere with the fragrance of (−)-ambrafuran. It has been reported that pyridine can be successfully replaced by other basic compounds, such as sodium hydride and sodium tert-amyl alcoholate. It has also been reported that (−)-ambrafuran can be obtained in 96% yield (75% overall from sclareol) by reacting the diol with butyllithium and tosyl chloride.

Another prior art method describes the carbonylation of allylic alcohols in the synthesis of an ambergris fragrance compound. Relevant in this process is the final step of the synthesis which involves the cyclisation to (−)-ambrafuran. The appropriate alcohol can be treated with a Lewis or Brönsted acid to achieve the cyclisation. A wide variety of acidic reagents were found to be able to result in the transformation. These reagents include boron trihalide and complexes thereof and several sulfonic acids. Trifluoromethanesulfonic acid, boron trifluoride and its complexes as well as alkyl- or arylsulfonic acids seem to be the preferred catalysts. The preferred solvents in which to carry out the cyclisation reaction seem to be halocarbon solvents at temperatures which vary from −110° C. to 150° C. At least 1, but up to 5 molar equivalents of the acidic cyclisation reagent need to be used.

U.S. Pat. No. 3,029,255 describes a method for achieving the cyclisation to (−)-ambrafuran by treatment of decahydro-2-hydroxy-2,5,5,8a-tetramethylnaphthaleneethanol with Al₂O₃ at 200-225° C., followed by heating in vacuo in the presence of β-naphthalene sulfonic acid (130-160° C.).

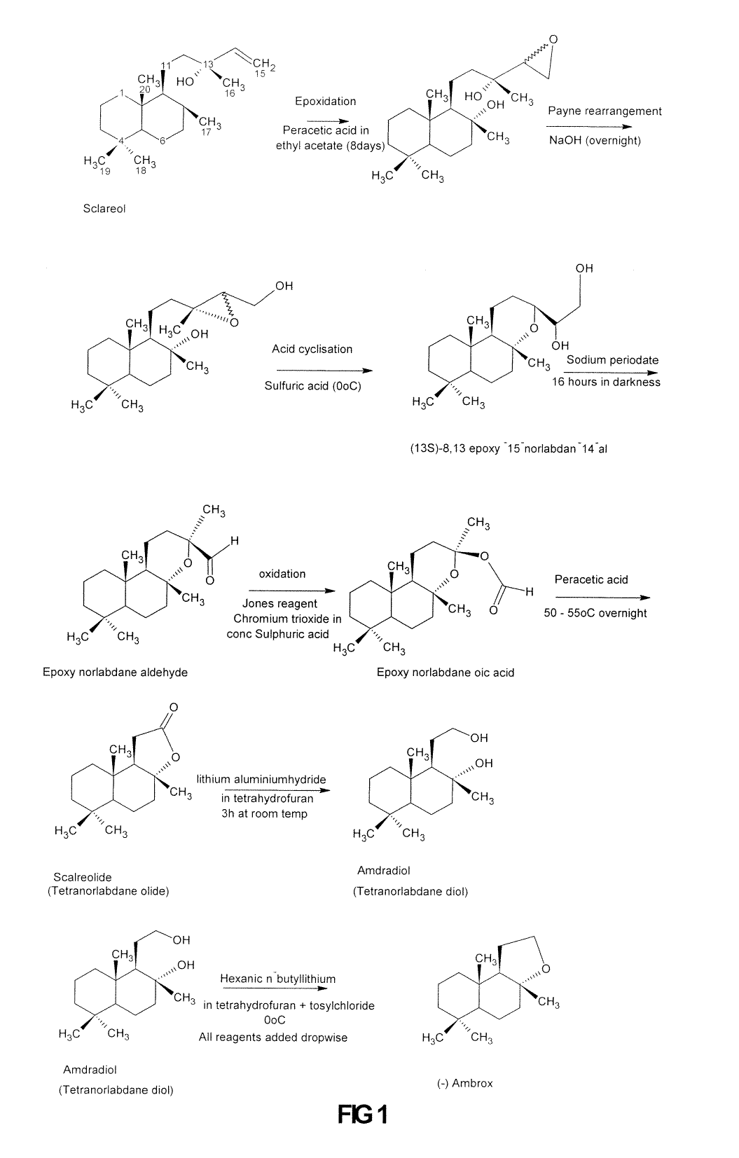

The synthesis of (−)-ambrafuran therefore involves approximately eight steps, some of which require the use of very harsh reagents which have to be disposed of in a very careful manner. FIG. 1 shows the conversion of sclareol to ambrafuran according to the prior art.

Zeolites are aluminosilicates which are generally used as solid acid catalysts. Because of their channel dimensions and stable structures these materials show unique selectivity and reactivity. Zeolites are environmentally benign and their use generally results in a reduction in waste and pollution. Zeolites, alumina and montmorillonite clay have been used for the catalytic dehydration of monoalcohols to ethers and olefins. The cyclodehydration of diols has also been used for the synthesis of heterocyclic compounds. Most of the cyclodehydration reactions using zeolites reported in the literature use very high temperatures (of the order of 175-225° C.). The present invention on the other hand provides a novel method for the cyclodehydration of a diol using a zeolite at room temperature with a total conversion of the diol to the cyclodehydrated product generally in less than two hours.

According to a first aspect of the invention there is provided a method for the cyclodehydration of a 1,4- or 1,5-diol, the method including the step of exposing a 1,4- or a 1,5-diol to an activated zeolite at a temperature of between about 0° C. and about 110° C. for a period of between about 1 and 24 hours, the activated zeolite being prepared from an inactive NaY type zeolite by ion exchange with an ammonium salt, to produce an ammonium zeolite and exchange of at least part of the ammonia of the ammonium zeolate with a group II A metal.

The NaY type zeolite may be that obtained from Zeolyst International (CBV100). Alternatively the calcium type zeolite (CBV320A) also obtained from Zeolyst International may be used.

In the case of the NaY-type zeolite, ion exchange with the ammonium salt is preferably carried out until the sodium level has been reduced¹. The Group II A metal is preferably calcium. The ion exchange of the ammonium cations with calcium is preferably carried out using calcium nitrate, although any suitable calcium salt may be used.

The cyclodehydration reaction may be carried out in a solvent such as toluene, ethyl acetate, diethyl ether, tetrahydrofuran or hexane.

In a preferred embodiment of the invention, the reaction may be carried out in a hydrocarbon or aromatic hydrocarbon solvent such as hexane or toluene at room temperature over a period of about 1 to 24 hours. Other C₅-C₉ hydrocarbon or aromatic hydrocarbon solvents may also be used.

The diol may be tetranorlabdane diol (or amdradiol). The product of the cyclodehydration may then be (−)-ambrafuran or Ambrox®.

According to a second aspect of the invention, there is provided a method of synthesing (−)-ambrafuran, the method including the microbiological conversion of sclareol to ambradiol followed by cyclodehydration to produce ambrafuran.

The microbiological conversion of the sclareol to ambradiol may be conducted with the micro organism Hyphozyma roseoniger.

The conversion of sclareol to a diol intermediate using the microorganism Hyphozyma roseoniger was described in 1989². The microorganism has the identifying characteristics of CBS214.83 and ATCC 20624. The organism was cultivated under aerobic conditions in an aqueous nutrient medium. Different forms of the organism could be used to achieve the conversion. These ranged from using the culture suspension, i.e. including the cells and the corresponding nutrient solution, or as suspended cells in a buffer solution, or even by immobilising the cells or an enzyme extract thereof on a solid support.

The aqueous medium for growing the organism may contain nitrogen sources, inorganic salts, growth factors, the desired substrate and additional carbon sources. When small amounts of yeast extract were added, supplementation with vitamins and trace minerals was not necessary. One or more trace minerals such as Fe, Mo, Cu, Mn and B could be added as well as vitamin B complex. The preferred temperature range for the cultivation of the microorganism was between about 18 and 28° C. with a pH between 3 and 6.5. The substrate range could vary between 1.5 and 30 g/l for optimum transformation. The substrate could be added to the medium as a powder or in the presence of an emulsifier such as Tween 80, as a slurry, or as a solution in an organic solvent such as acetone, ethanol or methanol. The organism was isolated from a soil sample from central New Jersey in the USA and deposited with CBS (Centraalbureau voor Schimmel Cultures) as well as with the ATCC (American Type Culture Collection).

The cyclodehydration step may be carried out using a Group II A metal zeolite. The Group II A metal may be calcium. The calcium zeolite may be a zeolite as described above. Instead the cyclodehydration step may be carried out in a hydrocarbon or aromatic hydrocarbon solvent such as hexane or toluene at room temperature or by dissolving ambradiol in a solvent such as dimethylsulphoxide (DMSO) or ethylacetate and optionally heating the solution.

For example the cyclodehydration may be conducted in DMSO at a temperature of between about room temperature and 180° C. Alternatively the cyclodehydration may be conducted in ethyl acetate at temperature of between about −20° C. to about 37° C.

The cyclodehydration step produces the (−)-isomer of ambrafuran i.e. Ambrox®. The starting material (sclareol) is a racemic mixture and the applicant believes that the microbiological oxidation of the racemic sclareol may be enantiomerically selective and produces a single enantiomer of ambradiol. However, the applicant has not ruled out the possibility that the desired enantiomer may be produced during the cyclodehydration step.

The invention is now described, by way of example, with reference to the following examples and the Figures, in which

FIG. 1 shows a reaction scheme for the synthesis of (−)-ambrafuran from sclareol;

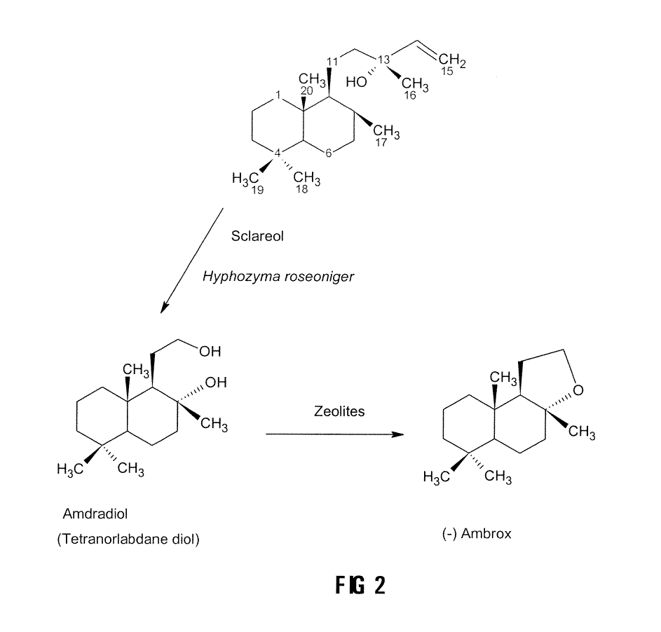

FIG. 2 shows a reaction scheme for the synthesis of (−)-ambrafuran from sclareol using the microorganism Hyphozyma roseoniger.

### EXAMPLE 1

### Analytical Methods

### (1) Quantitative Analysis of the Intermediate Diol and Ambrafuran

A Restek Rtx-5 sil w/intergra Guard, 0.25 mm ID. 0.25 μm film thickness (df) 30 meter GC column was used to analyse the conversion of the sclareol to ambradiol and the diol to ambrafuran. The GC program started at 180° C. and was increased to 270° C. at a rate of 15° C. per minute with a final run time of 6 minutes. The ambrafuran had a peak at 2.3 minutes, the diol at 3.6 minutes and the sclareol at 4.5 minutes. Calibration curves for the diol and (−) ambrafuran were also constructed.

(2) Chiral separation of (+) and (−)-ambrafuran

A Restek Rt-βDexsm 0.32 mmID. 0.25 μm df, 30 meter length was used to separate (+) and (−) ambrafuran. The temperature was held constant at 145° C. for 20 minutes. The (+) ambrafuran peak was at 17.3 minutes and the (−) ambrafuran at 16.42 minutes.

The structures of the diol and ambrafuran were confirmed by LC-MS

### EXAMPLE 2

### Production of the Intermediate Diol from Sclareol Using Hyphozyma roseoniger

### Reconstituting the Microorganism

The Hyphozyma roseoniger was purchased from ATCC in a freeze-dried powder form. It was reconstituted in sterile water and inoculated onto agar plates. The agar plates consisted of potato dextrose agar and the yeast cultivation medium. The plates were grown for 4 days at room temperature. The microorganism was streaked onto another set of plates to ascertain purity of the culture. It was then inoculated into broth consisting of the yeast cultivation medium. It was grown for 3 days and the cells were spun down and re-suspended in a minimal volume of 100 mM potassium phosphate buffer, pH6.5. The cell suspension was mixed with an equal volume of 50% glycerol and then placed into cryovials as the master cell bank.

All experiments were carried out using a vial from the master cell bank. Microorganism (500 μl) was inoculated into 10 ml of either potato dextrose broth (PDB), the PDB plus substrate (sclareol), malt extract or malt extract plus substrate, nitrogen base or nitrogen base plus substrate. The cultures were grown for 3 days at room temperature and agitation at 180 rpm.

The microorganism (5 ml into 100 ml) was then inoculated into different media containing substrate (0.02%). The different media could be selected from nitrogen base, potato dextrose broth plus nitrogen base and nitrogen base plus malt extract.

In one set of experiments, the microorganism was first grown in potato dextrose broth plus nitrogen base or malt extract and nitrogen base for 3 days without substrate. The cells were harvested and then re-suspended in 100 mM potassium phosphate buffer pH 6.5 and the substrate was added.

Samples were taken every 24 hours for 5 days and analysed for the formation of the intermediate diol and any unwanted peaks.

Different substrate concentrations were also tested ranging from 10 mg/100 ml to 20 g/100 ml.

To improve the substrate concentration for maximum productivity, a set of experiments was done in which the microorganism was grown in yeast nitrogen base without amino acids containing the substrate (0.02%) and Tween 80 (500 μl/100 ml). Substrate (0.5 g) mixed with 0.5 g of Tween 80 was then added every 24 hours for 5 days and the conversion to diol was monitored.

The preferred conditions were to grow the microorganism as normal for 3 days with 0.02% substrate and then 1 g of substrate mixed with 1 g of Tween 80/100 ml was added and monitored for 8 days for conversion. Scaled-up reactions were carried out in which 10 g to 15 g of sclareol and 10 ml Tween 80 were added to a 1 L reaction mixture. The preferred temperature for the conversion of sclareol to intermediate diol was 20° C. The temperature range was between 18° C. and 32° C.

Following full conversion of the sclareol to the diol, the diol was extracted from the mixture by addition of ethyl acetate, separated from the aqueous phase and dried over anhydrous magnesium sulphate and the solvent removed under reduced pressure.

The media described above for cultivating the microorganism as well as converting the substrate all gave good conversion, but the yeast nitrogen base without amino acids with substrate gave the intermediate diol (>98% yield) without any by-products.

### EXAMPLE 3

### Preparation of the Zeolites

Inactive zeolites NaY type from Zeolyst (25 g) was mixed with 250 ml 10% ammonium nitrate and ion exchanged by refluxing at 90° C. for 24 hours. The mixture was filtered, washed with distilled water and dried overnight at 105° C. The procedure was repeated with 10% calcium nitrate and the zeolite was then activated at approximately 500° C. under vacuum.

Alternatively the Zeolite CBV320 (CaY type) can be purchased from Zeolyst International in the inactivate form and can be activated under vacuum at 500° C.

Yet another alternative was to activate the Zeolite CBV320 in a conventional microwave oven. The preferred method was to activate 50 g of Zeolite CBV320 by heating at 500 W for 15 minutes in an open container in the microwave. The Zeolites were allowed to cool and then kept in a closed container

### EXAMPLE 4

### Conversion of the Diol to (−)Ambrafuran

The conversion of the diol to the (−) ambrafuran was accomplished by cyclodehydration. Approximately 10 to 50 mg of the intermediate diol, prepared from sclareol with the Hyphozyma roseoniger microorganism as described above was, in different embodiments of the invention, dissolved in 10 ml of toluene, ethyl acetate, diethyl ether, ethanol or hexane and placed in a round bottom flask. Activated zeolite (10 mg to 500 mg), prepared as described above, was added and the mixture was allowed to react at temperatures ranging from room temperature to 110° C. for 1 to 24 hours. The results are set out in Table 1 below.

TABLE 1
Conversion of the diol to (−) ambrafuran with different
solvents in 4 hours at room temperature

| Solvent | Conversion (4 hours) |
|---|---|
| Diethyl ether | 25 |
| Ethanol | 5.4 |
| Ethyl acetate | 3.7 |
| Toluene | 100 |
| Hexane | 100 |

In another embodiment of the invention, the reaction was carried out at room temperature for 1 to 4 hours in toluene with a ratio of 400 mg diol to 20 ml toluene and 1:4 to 1:9 diol to activated zeolite. With the toluene and activated zeolite, full conversion was achieved in 1 to 24 hours at room temperature without the formation of any by-products. The product in each case was the (−)-enantiomer, (−)-ambrafuran, as shown by GC. The applicant believes that the (−)-enantiomer is produced in the microbiological conversion of racemic sclareol by the Hyphozyma roseoniger to produce an optically active diol².

The preferred method was to dissolve 400 mg of the intermediate diol in 20 ml hexane with 1.6 to 3.6 g (1:4 to 1:9 ratio) of activated CBV320 zeolites. The mixture was allowed to react at room temperature for 2 to 24 hours. The zeolites were removed with centrifugation at 3000 rpm for 5 minutes. The zeolites were washed with warm hexane or warm ethanol to remove any product associated with the zeolites. The hexane was removed under reduced pressure to yield a final product (−) Ambrafuran with a purity of at least 96% and yield of 98%.

The reaction could be conducted with or without a nitrogen blanket. The zeolite was filtered off or the mixture centrifuged to remove the zeolite and the solvent was removed under reduced pressure.

In other embodiments, the cyclodehydration was carried out in DMSO. Approximately 10 mg of the diol was dissolved in 10 ml DMSO dried on molecular sieves (4 Å). In different embodiments, the reaction was run at temperatures ranging from room temperature to 180° C. under a nitrogen blanket. In a preferred embodiment, the temperature was about 180° C.

In other embodiments the diol was dissolved in ethyl acetate at temperatures from between −20° C. and 37° C. for approximately 2 weeks. This also resulted in conversion of the diol to the (−)-ambrafuran.

### DISCUSSION

The reaction accordingly provides a novel process for the cyclodehydration of ambradiol to (−)-ambrafuran using activated zeolites (activated at 500° C. under vacuum or in a conventional microwave) at room temperature using a hydrocarbon solvent such as hexane or toluene. It also provides a novel process for producing (−)-ambrafuran from ambradiol in a cyclodehydration using DMSO at elevated temperatures. It also provides a method of producing (−)-ambrafuran from racemic sclareol by microbiological conversion of sclareol to ambradiol using Hyphozyma roseoniger followed by cyclodehydration to (−)-amberfuran. The invention further provides an activated zeolite by activation of an inactive zeolite (NaY type) by ion exchange with ammonium nitrate followed by ion exchange with calcium nitrate followed by high temperature drying or use of the calcium zeolite CBV320. The zeolites used in the method of the invention can be re-activated merely by heating at 500° C. under vacuum or at 500 W in a conventional microwave oven.

The invention thus provides a new process for the cyclodehydration of a diol precursor for the synthesis of the ambergris compound (−)-ambrafuran as well as an efficient two step process for the complete synthesis of (−)-ambrafuran from sclareol by the conversion of sclareol to an intermediate diol using a microorganism and cyclodehydration of the intermediate diol to (−)-ambrafuran.

### REFERENCES

(1) Y Kanno, Y Matsui, H Imai (1985). Mechanistic model of disproportionation of nitrogen monoxide on CaHY-type sodium. journal of Inclusion Phenomena 3, 461-469.
(2) M I Farbood, B J Willis (1989). Process for producing diol and furan and microorganism capable of same. U.S. Pat. No. 4,798,799.

1. A method for the cyclodehydration of a 1,4- or 1,5-diol, the method including the step of exposing a 1,4- or a 1,5-diol to an activated zeolite at a temperature of between about 0° C. and about 110° C. for a period of between about 1 and 24 hours, the activated zeolite being prepared from an inactive NaY or CaY type zeolite by ion exchange with an ammonium salt, to produce an ammonium zeolite and exchange of at least part of the ammonia of the ammonium zeolate with a group II A metal.

2. A method as claimed in claim 1, in which the Group II A metal is calcium.

3. A method as claimed in claim 1, in which the ion exchange of the ammonium cations with calcium is carried out using calcium nitrate.

4. A method as claimed in claim 1, in which the cyclodehydration reaction is carried out in a solvent selected from toluene, ethyl acetate, diethyl ether, tetrahydrofuran, hexane and mixtures thereof.

5. A method as claimed in claim 4, in which the reaction is carried out in toluene at room temperature over a period of between about 1 and 24 hours.

6. A method as claimed in claim 4 in which the reaction is carried out in hexane at room temperature over a period of between 1 to 24 hours.

7. A method as claimed in claim 1, in which the diol is tetranorlabdane diol (or amdradiol).

8. A method of synthesing (−)-ambrafuran, the method including the microbiological conversion of sclareol to ambradiol followed by cyclodehydration to produce ambrafuran.

9. A method as claimed in claim 8, in which the microbiological conversion of the sclareol to ambradiol is carried out using the micro organism Hyphozyma roseoniger.

10. A method as claimed in claim 8, in which the cyclodehydration step is carried out using a Group II A metal zeolite.

11. A method as claimed in claim 10, in which the Group II A metal is calcium.

12. A method as claimed in claim 8, in which the cyclodehydration step is carried out by dissolving ambradiol in a solvent and optionally heating the solution.

13. A method as claimed in claim 12, in which the solvent is selected from hydrocarbon and aromatic hydrocarbon solvents and the reaction is carried out at room temperature.

14. A method as claimed in claim 13, in which the hydrocarbon and aromatic hydrocarbon solvents are selected from hexane and toluene.

15. A method as claimed in claim 12, in which the solvent is selected from dimethylsulphoxide (DMSO) and ethylacetate.

16. A method as claimed in claim 15, in which the cyclodehydration is conducted in DMSO at a temperature of between about room temperature and 180° C.

17. A method as claimed in claim 15, in which the cyclodehydration is conducted in ethyl acetate at a temperature of between about −20° C. and 37° C.
